(19) United States
(12) Patent Application Publication
(10) Pub. No.: US 2006/0160200 A1
(43) Pub. Date: Jul. 20, 2006

(54) Supporting body with immobilized catalytically active units

(76) Inventors: Jorg Rathenow, Eppstein (DE); Jurgen Kunstmann, Bad Soden (DE); Andreas Ban, Koblenz (DE); Soheil Asgari, Wiesbaden (DE)

Correspondence Address: DORSEY & WHITNEY LLP;INTELLECTUAL PROPERTY DEPARTMENT, 250 PARK AVENUE, NEW YORK, NY, 10177, US

(21) Appl. No.: 11/343,479

(22) Filed: Jan. 30, 2006

Related U.S. Application Data
(63) Continuation-in-part of application No. PCT/EP04/08641, filed on Aug. 2, 2004.

(30) Foreign Application Priority Data
Jul. 31, 2003 (DE) DE 103 35 130.2

(51) Int. Cl. C12N 11/02; C12M 1/14

(52) U.S. Cl. 435/177; 422/222; 435/299.1

(57) ABSTRACT

The present invention relates to the use of porous carbon-based bodies for the support and/or immobilization of catalytically active units. Catalytically active units for chemical and/or biological reactions may be essentially immobilized on such supporting bodies. The catalyst units can comprise catalytically active units and porous carbon-based supporting bodies. The present invention further relates to reactors comprising these catalyst units and their use in chemical and biological reactions.

### CROSS-REFERENCE TO RELATED APPLICATIONS

This application is a continuation-in-part application of International Patent Application No. PCT/EP2004/008641, filed Aug. 2, 2004, which claims priority from PCT Patent Application No. PCT/EP2004/000077, filed Jan. 8, 2004, and from German Patent Application No. DE 103 35 130.2, filed Jul. 31, 2003, the entire disclosures of which are incorporated herein by reference.

### BACKGROUND OF THE INVENTION

Many chemical and biological reactions may be carried out on an industrial scale using catalysts. Catalysts can reduce the activation energy, allow for the selective execution of reactions, and thereby may improve the economy of a given process. Many kinds of compounds, from simple organometallic complexes to enzymes that are built in a complex manner, may be utilized as catalysts.

Reactions on an industrial scale can require high throughputs and may be subject to economical considerations. In order to be able to better separate the catalysts from the product mixture, or in order to be able to reuse them subsequently, catalysts can be immobilized on solid substrates. The catalysis can occur at the interface between the reaction medium and the substrate that is loaded with catalytically active units (or “catalytic units”). The immobilization of the catalytic units can also allow for a continuous catalyzed process without a continuous addition of catalyst.

In addition, methods employing immobilized catalytic units can utilize high catalyst concentrations, so that high reaction rates and smaller dimensioned systems may be possible. The duration of the process also may be shortened significantly. With immobilized enzymes, for example those used in fermentation processes, higher reaction rates can be achieved than when using free enzymes. International Patent Publication WO 00/06711, for example, describes the immobilization of enzymes using diatomaceous earth as supporting material.

The aforementioned method and other conventional methods for supporting catalysts often have certain disadvantages. For example, the supports may not be modifiable in any desired way, or the supporting material may have an inferior compatibility, or the immobilization process may involve high losses of catalytic material or activity.

One of the objects of the present invention is t provide exemplary immobilized “catalyst units” that overcome the disadvantages mentiuoned above. Preferably, these immobilized catalyst units can be suitable for reactions on an industrial scale. This can be accomplished with the use of porous carbon-based bodies as supporting materials.

### SUMMARY OF THE INVENTION

The present invention relates to the use of porous carbon-based bodies for the support and/or immobilization of catalytically active units. The present invention further relates to porous carbon-based supporting bodies that may have a layer-like construction comprising at least two porous material layers that can be provided adjacent to each other, between which a region may exist that allows flow therethrough. At least one porous material layer may be provided that, while keeping its shape, can be rolled up onto itself or arranged in such a way that a region that may allow flow therethrough exists between at least two adjacent sections of the material layer. The present invention further relates to catalytically active units for chemical and/or biological reactions that may be immobilized on such supporting bodies. The exemplary catalyst units may comprise catalytically active units and porous carbon-based supporting bodies, and reactors comprising these catalyst units may be used in chemical and biological reactions.

The present invention further relates to the use of a carbon-based porous body for the support and/or immobilization of catalytic units used in chemical and/or biological reactions.

The present invention further relates to catalytic units, as well as reactors which may comprise a porous carbon-based supporting body and catalytic units.

The present invention further relates to reactors for chemical or biological reactions that comprise one or more catalytic units.

In one exemplary embodiment of the present invention, a supporting body can be provided comprising at least one carbon-based porous material layer that may be rolled up onto itself or arranged to form a cylindrical body, such that one or more spaces are formed between at least two adjacent sections of the at least one porous material layer that are capable of supporting flow.

Further exemplary embodiments of the present invention relate to a catalyst unit comprising catalytically active units and porous carbon-based supporting bodies. In certain exemplary embodiments, the catalytically active units may be affixed to the supporting bodies.

Still further exemplary embodiments of the present invention relate to a porous carbon-based supporting body comprising a plurality of material layers.

In yet further exemplary embodiments of the present invention, the supporting bodies may comprise a plurality of channels. The channels may be approximately parallel, and may further have a linear, wave-like, meandering, or zigzag-shape within a layer.

In certain exemplary embodiments of the present invention, the porous carbon-based supporting body may be produced by carbonization of a sheet material which may further be structured, rolled, embossed, pre-treated, or folded. The sheet material can comprise at least one of fiber, paper, textile, or polymer material.

In still further exemplary embodiments of the present invention, the outer surface of the supporting body may be at least partially in direct contact with a semipermeable separating layer that can be essentially impermeable to the catalytically active units.

In still further exemplary embodiments of the present invention, the porous carbon-based supporting body may be arranged in a housing, or in or on a suitable container. The suitable container an be a flask, a bottle, a chemical reactor, a biological reactor, a stirred reactor, a fixed bed reactor, a fluid bed reactor, or a tubular reactor. At least part of the wall of the container may comprise a semipermeable separating layer that can be approximately impermeable to the catalytically active units.

Further exemplary embodiments of the present invention relate to a reactor for chemical or biological reactions comprising one or more catalyst units, which further comprise a carbon-based porous supporting body and one or more catalytically active units which may comprise organometallic complex compounds, metals, metal oxides, alloys, or enzymes. The reactor may further comprise a chamber located within the reactor, wherein at least part of the chamber wall may comprise a semipermeable separating layer that can be essentially impermeable to the catalytically active units, with one or more supporting bodies optionally located within the chamber.

Exemplary embodiments of the present invention are described by, ascertained from and/or encompassed by, the description provided herein.

### BRIEF DESCRIPTION OF THE DRAWINGS

The following description, given by way of example, but not intended to limit the invention solely to the specific exemplary embodiments described, may best be understood in conjunction with the accompanying drawings, in which:

FIGS. 1A-1C are schematic illustrations of one exemplary embodiment of the present invention comprising supporting bodies having a layer-like construction.

FIGS. 2A and 2B are schematic illustrations of another exemplary embodiment of the present invention comprising cylindrical supporting bodies having a circular surface that may be exposed to the flow of reactants.

### DETAILED DESCRIPTION OF EXEMPLARY EMBODIMENTS

Exemplary Definitions

The term “catalytic unit(s)” may comprise catalytically active substances, in particular metals, metal compounds, alloys, organometallic complexes, or enzymes, and may exclude living cells or organisms or cells and organisms that are capable of multiplication or reproduction.

The term “porous carbon-based supporting body” may be understood to be, but is not limited to, porous bodies that comprise carbon-containing material, including carbides, and which further may comprise carbon, and which may have a certain average pore size. These bodies may serve as supporting material for catalytic units.

The term “semipermeable separating layer” may be understood to be, but is not limited to, a layer that may be in direct contact with the porous body, and which may be either impermeable with respect to the catalytic units and permeable to the respective reaction products and educts as well as the reaction medium, or which may be impermeable to the catalytic units and the products and permeable to the respective educts and the reaction medium.

The term “catalyst unit” may be understood to mean, but is not limited to, a porous supporting body comprising catalytic units and which may optionally have its outer surface in direct contact with a semipermeable membrane, and which further may be sealed or arranged in a housing.

The term “chemical reactions” may be understood to comprise, but is not limited to, reactions that can be achieved without the utilization of living organisms or cells or organisms or cells that are capable of multiplication or reproduction.

The term “biological reactions” may comprise, but is not limited to, reactions utilizing enzymes, and may exclude those utilizing living cells or organisms or cells and organisms that are capable of multiplication or reproduction.

The term “reaction medium” may comprise, but is not limited to, any fluid, gaseous or liquid, including but not limited to water, organic solvents, inorganic solvents, supercritical gases, as well as conventional carrier gases.

The term “educt” may comprise, but is not limited to, the starting materials of a chemical or biological reaction or, in the case of biological reactions, it may comprise nutrients, oxygen, and optionally carbon dioxide.

The term “product” may be understood to include, but is not limited to, reaction products of a chemical reaction, or the reactions products or conversion products in case of biological or enzymatic reactions.

The term “reaction mixture” may be understood to include, but is not limited to, a mixture comprising the reaction medium, other reactants, and may optionally comprise educts and/or products.

Supporting Bodies and Catalyst Units

In certain exemplary embodiments of the present invention, porous carbon-based supporting bodies may be used as supporting material for the immobilization of catalytic units. Catalyst units may be obtained by at least partial sealing of individual outer surfaces of these porous supporting bodies, or by arranging these bodies in suitable housings or containers. Catalyst units in certain exemplary embodiments of the present invention may be usable as exchangeable cartridges in cartridge systems or in suitable reactors.

Porous carbon-based supporting bodies may be dimensionally stable and may vary with respect to their construction, including such features as pore sizes, internal structure, and outer overall shape. By varying these properties, the porous carbon-based supporting bodies may be tailored to a plurality of applications.

In the description provided herein, “carbon-based” may be understood to designate materials that, prior to a potential modification with metals or other compositions, may have a carbon content of more than 1% by weight, more than 50% by weight, more than 60% by weight, more than 70% by weight, more than 80% by weight, or optionally more than 90% by weight. In certain exemplary embodiments of the present invention, the carbon-containing supporting bodies may contain between 95% and 100% by weight of carbon, or between 95% and 99% by weight of carbon.

The porous supporting bodies may comprise activated carbon, sintered activated carbon, amorphous, vitreous, crystalline, or semicrystalline carbon, graphite, carbon-containing material that was produced pyrolytically or by means of carbonization, carbon fibers, or carbides, carbonitrides, oxycarbides or oxycarbonitrides of metals or nonmetals, as well as mixtures thereof. In certain exemplary embodiments of the present invention, the porous bodies may comprise amorphous and/or pyrolytic carbon prior to being optionally modified with metals.

Porous supporting bodies may be produced by means of pyrolysis or carbonization of starting materials that are converted to the aforementioned carbon-containing materials under high temperature in an oxygen-free atmosphere. Suitable starting materials for carbonization include, but are not limited to, polymers, polymer films, paper, impregnated or coated paper, wovens, nonwovens, coated ceramic disks, cotton wool, cotton swabs, cotton pellets, cellulose materials, or, optionally legumes such as peas, lentils, beans and the like, nuts, dried fruits and the like, or green bodies produced on the basis thereof.

In certain exemplary embodiments of the present invention, the porous body may further comprise substances, doping agents, additives, and/or co-catalysts selected from organic and inorganic substances or compounds. Substances such as compounds of iron, cobalt, copper, zinc, manganese, potassium, magnesium, calcium, sulfur, or phosphorus may be used.

For enzymatic or biological reactions, the porous body may be impregnated with a coating comprising carbohydrates, lipids, purines, pyromidines, pyrimidines, vitamins, proteins, growth factors, amino acids, and/or sulfur or nitrogen sources.

The average pore size of the porous body may be between about 2 Å and 1 millimeter, preferably between about 1 nm and 400 μm, or between about 10 nm and 100 μm.

The porous carbon-based bodies according to certain exemplary embodiments of the present invention may have a layer-like construction comprising:
i) at least two porous material layers that are arranged adjacent to each other and which may be connected with one another, and between which a region exists that may allow flow therethrough; or
ii) at least one porous material layer that, while keeping its shape, may be rolled up onto itself or arranged in such a way that a region exists between at least two adjacent sections of the material layer that that may allow flow therethrough.

The supporting body may comprise a plurality of material layers arranged adjacent to each other, wherein intermediate regions may exist between some or all of the material layers that may allow flow therethrough. Each such region may comprise channel-like structures, for example, a plurality of channels, which may run essentially parallel to one another, which may be crossed, or which may be networked. The channel-like structures may be provided by means of a plurality of spacing elements that are arranged on the supporting material layers and which may keep them separated to a certain degree. The channels or channel-like structures may have average channel diameters in the range of about 1 nm to about 1 m, or about 1 nm to about 10 cm, preferably about 10 nm to 10 mm, and more preferably about 50 nm to 1 mm. The distance between any two adjacent material layers may be uniform or nearly identical. However, different distances may also be used between different adjacent layers or in between different areas of the same two layers.

The exemplary supporting body according to the exemplary embodiment of the present invention may be constructed in such a way that it comprises channels between a first layer and a second material layer which are approximately parallel to channels between the second layer and a third material layer, such that the supporting body overall comprises channel layers that allow flow therethrough in a preferred direction. Alternatively, the exemplary supporting body may also be designed in such a way that channels between a first layer and a second material layer are configured at a particular angle or range of angles with respect to the channels between the second material layer and a third material layer, wherein the angle may be greater than 0° and up to 90°, or preferably about 30° to 90°, or more preferably about 45° to 90°, such that the supporting body comprises a plurality of channel regions that are angularly offset with respect to one another.

The channels or channel-like structures in the supporting body according to certain exemplary embodiments of the present invention may be open at both ends, such that the body has a kind of “sandwich structure” comprising regions of porous material layers alternating with regions in-between that allow flow therethrough and which may further be configured as channels. Such channels or channel-like structures may extend linearly in a longitudinal direction, or alternatively they may be wave-like, meandering, zigzag, or in other directions. Within a given region between two porous material layers such channels may be approximately parallel or they may intersect.

The outer shape and dimensioning of the supporting body may be chosen based on the intended application. The outer shape of the supporting body may be selected, for example, from elongated shapes, including but not limited to cylindrical shapes, polygonal columnar shapes such as triangular columns or ingot shapes, plate-like shapes, polygonal shapes such as square, cuboidal, tetrahedral, pyramidal, octahedral, dodecahedral, icosahedral, rhombohedral, prismatic and the like, or generally round shapes including spherical, hollow ball-shaped, spherically or cylindrically lens-shaped, disk-shaped or ring-shaped.

Supporting bodies according to certain exemplary embodiments of the present invention may have overall dimensions that are selected based on the intended application. For example, supporting body volumes may be approximately 1 mm³, or about 1-10 cm³, or up to about 1 m³. The supporting bodies may also be significantly larger or smaller than these exemplary volumes, depending on the requirements of the desired application. The supporting body may have a largest outer dimension in the range of about 1 nm to 1,000 m, preferably about 0.5 cm to 50 m, or about 1 cm to 5 m. The dimensions of the supporting body need not be limited by these ranges, and may be chosen based on the requirements of a particular application.

In one exemplary embodiment of the present invention, the supporting body may be disk-shaped or cylindrical, and may have a diameter in the range of about 1 nm to 1,000 m, preferably about 0.5 cm to 50 m, or more preferably about 1 cm to 5 m. A cylindrical or disc-shaped supporting body may be formed, for example, by rolling up a material layer, which may optionally be corrugated, embossed, or otherwise structured, such that a region that may allow flow therethrough exists between at least two adjacent sections of the material layer. Such flow-through regions may comprise a plurality of channel-like structures or channels. In other exemplary embodiments, several material layers that are adjacent or stacked on top of one another may also be formed into cylindrical supporting bodies by rolling the layers up.

The porous material layers and/or the channel walls or spacing elements between the material layers of supporting bodies may have average pore sizes in the range of about 1 nm to 10 cm, preferably about 10 nm to 10 mm, and more preferably about 50 nm to 1 mm. The porous material layers optionally may be semipermeable and may have a thickness of between about 3 Å and 10 cm, or preferably from about 1 nm to 100 μm, or more preferably about 10 nm to 10 μm. The average pore diameter of the porous, optionally semipermeable, material layers may be between about 0.1 Å and 1 mm, preferably from about 1 Å to 100 μm, or more preferably about 3 Å to 10 μm.

The catalytic units fixed or essentially immobilized on the supporting body may comprise catalytically active substances, including metals, metal compounds, alloys, organometallic complexes, and enzymes, and may exclude living cells or organisms or cells and organisms that are capable of multiplication or reproduction. Such catalytic units may comprise catalytically active metals, alloys or metal compounds selected from the main group and auxiliary group metals of the periodic system of the elements, including transition metals such as Sc, Y, Ti, Zr, Hf, V, Nb, Ta, Cr, Mo, W, Mn, Re, Fe, Ru, Os, Co, Rh, Ir, Ni, Pd, Pt, Cu, Ag, Au, Zn, Cd, or Hg, as well as the lanthanides and actinides; alloys and compounds thereof, or organometallic complex compounds. In certain embodiments of the present invention, Ga, In, Tl, Ge, Sn, Pb and Bi may be preferred main group metals, as well as alloys and compounds thereof, or also organometallic complex compounds.

Catalytic units may be applied to the supporting body using conventional methods, for example by means of vacuum deposition of the metal or metal compound vapor, sputtering, or spraying or dipping methods using solutions, emulsions, or suspensions of the metals, alloys, or metal compounds in suitable solvents or solvent mixtures.

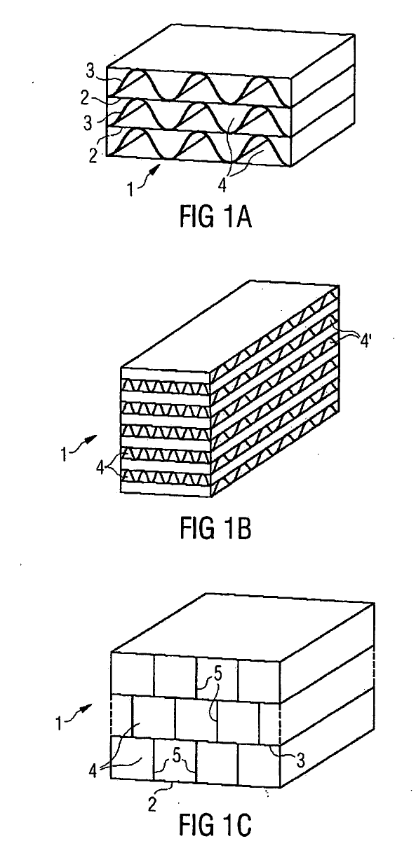

FIGS. 1A-1C illustrate layer-like constructions of the supporting bodies according to certain exemplary embodiments of the present invention. A supporting body 1 shown in a perspective view in FIG. 1A comprises material layers 2 and 3 that are arranged in alternating order. In this exemplary embodiment, first material layer 2 adjoins and may be connected with a second material layer 3, which may optionally be structured (e.g., corrugated or folded). A region may thus be formed between the material layers 2 and 3 that comprises a plurality of parallel channels 4, which can permit flow therethrough. In one exemplary embodiment of the present invention, the supporting body of FIG. 1A may have a structure similar to that of a corrugated cardboard stack. Alternatively, the structured material layers may be arranged in layers having an alternating angular offset. If the angular offset is approximately 90°, an exemplary supporting body such as that depicted in FIG. 1B results, wherein the flow may occur crosswise through channels 4 and 4′. Other offset angles may also be used. The exemplary supporting body illustrated in FIG. 1B is essentially open on its frontal surfaces. Because of the crosswise alternating corrugated structure layers, this supporting body can comprise two possible flow-through directions that are angularly offset with respect to each other. In another exemplary embodiment of the present invention illustrated in FIG. 1C, two or more substantially flat or planar material layers 2, 3 may also be arranged adjacent to each other, and these material layers may further be connected by means of spacing elements 5. In this exemplary configuration, a plurality of channels is present in the regions between the material layers 2, 3 that may allow flow therethrough.

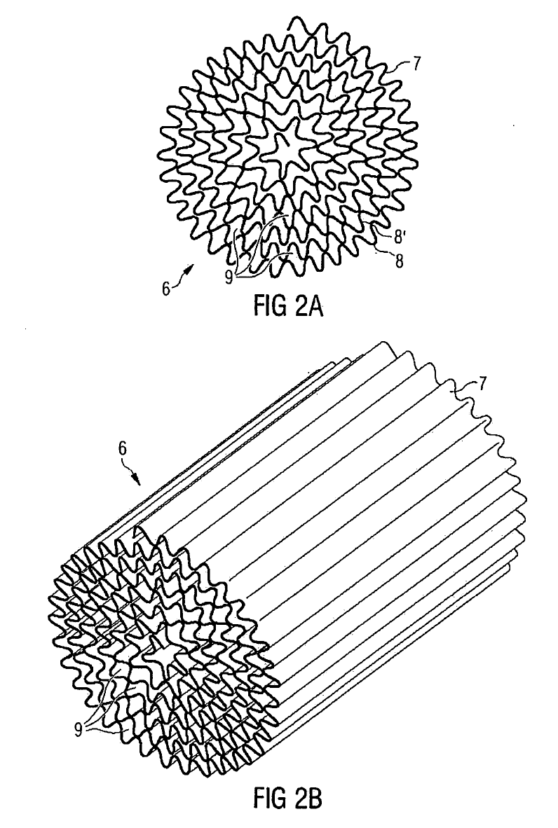

FIGS. 2A and 2B illustrate further exemplary embodiments of supporting bodies of the present invention. For example, FIG. 2A shows a top view of cylindrical supporting body 6 comprising corrugated material layer 7 that is rolled up in a spiral shape. Using spiral winding, a plurality of regions may be formed between a section 8 of the material layer and a further section 8′ in the adjacent winding, such that interstitial channels 9 are present between sections 8 and 8′. As can be seen in FIG. 2B, the exemplary supporting body 6 may be cylindrically constructed by winding or rolling up of a sheet-like material having a wave-like structure or pattern. Supporting cylindrical bodies may be formed, for example, by rolling up a sheet of corrugated cardboard or similar material. Using carbonization of the corrugated cardboard material, cylindrical formed pieces 6 may be obtained, wherein a plurality of channels 9 are formed approximately parallel to the cylinder axis. The resulting cylindrical supporting body 7 has an approximately circular face, as shown in FIG. 2A, and allows uniaxial flow approximately parallel to the axis of the cylinder.

In an exemplary embodiment of the present invention, the material layers of the supporting body may be structured on one or both sides thereof. The structure of the material layers may be in the form of a corrugation of the material layer, or alternatively in the form of an impressed or otherwise formed groove pattern, whereas the grooves or channel-like depressions may be arranged essentially equidistant to each other over one or more material layers. Groove patterns may run parallel to the outer edges of the material layers, may be arranged in any angle thereto, may have zigzag patterns and/or may have wave-like patterns. The material layers, if structured on both sides, may have similar or different groove patterns on opposite sides of a layer. In certain exemplary embodiments of the present invention, the porous material layers may have a uniformly complementary structure on opposite sides, that is, the groove impressions on one side of the material layer correspond to a heightened protuberance on the directly opposite side of the material layer. The material layers in the supporting body may be arranged in such a way that the groove patterns of two adjacent material layers runs essentially parallel to each other.

The material layers may also be arranged in such a way that the groove patterns or corrugations of two adjacent material layers lie at an angle with respect to each other, such that a plurality of contact points may be formed between the adjacent material layers at the positions where raised edges or portions of opposing groove structures corresponding to the adjacent material layers meet. In this manner, the exemplary supporting bodies may be obtained that have a significantly increased mechanical stability as a result of the connections formed at many locations corresponding to the contact points of intersecting groove patterns. The groove structures may be selected in such a way that a channel or network-like structure results, corresponding to a plurality of channels or tubes, in the intermediate regions between two material layers that are configured adjacent to one another. Such exemplary configurations may lead to a reduced flow resistance in the supporting body.

In alternative exemplary embodiments of the present invention, the material layers may be pre-formed in a corrugated manner, or folded in a zigzag or harmonica-like manner, rather than or in addition to comprising grooves or embossed channels. Arranging several such material layers on top of one another other can produce comb-like structures as viewed from one end that comprise channel structures in the direction of the material layer planes. When such pre-formed material layers are rolled up, cylindrical supporting bodies result, the cross-section of which may exhibit a plurality of spirally arranged channels that extend parallel to the longitudinal axis of the cylinder. Such cylinders or disks may be essentially open on both ends, permitting flow therethrough approximately parallel to the cylindrical axis.

In further exemplary embodiments of the present invention, spacing elements may alternatively or additionally be positioned or provided between adjacent material layers. Such exemplary spacing elements may provide larger spaces between the material layers, and may help to form channels between the material layers, thereby providing a low flow resistance. Spacing elements may comprise porous or open-pore sheet materials having the form of intermediate layers, network structures, or alternatively they may be spacers arranged at the edges of the material layers or centrally, thereby providing a certain minimum distance between adjacent material layers.

The supporting bodies according to certain exemplary embodiments of the present invention may exhibit intermediate layers or channels or channel layers that are essentially or approximately open at both ends of the channels or layers. Supporting bodies may preferably be open and not sealed against fluids on the front and/or edge sides of the material layers, or at the entrances or exits of the channels.

A plurality of channel-like structures may be formed by using groove embossings, foldings, or corrugations of particular dimensions wherein these features may be arranged at certain relative angles between adjacent material layers and provide, as described above, a plurality of contact points. Alternatively, such channel-like structures may also be accomplished by providing nearly parallel folds or corrugations in adjacent material layers that have different widths.

The material layers may also be separated by providing alternating groove embossings or foldings or corrugations having different depths on the material layers. Such features may be characterized by varying elevations or heights of individual groove edges, such that the number of actual contact points between adjacent material layers at the positions of intersecting edges of the grooves, corrugations, or folding structures overall may be decreased relative to the total number of groove edges present. By connecting the material layers at these positions, mechanical strength and a low flow resistance may be provided in the supporting body.

In other exemplary embodiments of the present invention, porous supporting bodies having a modular structure may be provided by carbonization of an optionally structured, embossed, pre-treated, or folded sheet material comprising fiber, paper, textile, or polymer material. Such supporting bodies may comprise a carbon-based material, or optionally a carbon composite material, that may be produced by pyrolysis of carbon-containing starting materials and which further may comprise carbon ceramics or carbon-based ceramics. Suitable materials may be produced, for example, by pyrolysis or carbonization of paper-like starting materials at high temperatures. A production of carbon composite materials is described, for example, in International Patent Publication WO 01/80981. The exemplary carbon-based supporting bodies may further be produced using methods such as those described in International Patent Publication WO 02/32558.

The exemplary supporting bodies may also be provided by pyrolysis of suitably pre-produced polymer films or three-dimensionally arranged or folded polymer film packets as described, for example, in German Patent Application DE 103 22 182.

In other exemplary embodiments of the present invention, pyrolysis methods such as those described above may be used to provide supporting bodies by carbonization of corrugated cardboard, wherein the corrugated cardboard layers may be fixed atop one another in a suitable manner prior to carbonization, so that an open body results which may permit flow therethrough.

Supporting bodies in cylindrical form may also be provided by rolling up or winding of paper or polymer film layers or stacks, which may be arranged in parallel or in a cross flow configuration, into cylindrical bodies, tubes, or rods, followed by pyrolysis thereof in accordance with the methods described above.

In certain exemplary embodiments of the present invention, these “wound bodies” may comprise a grooved, embossed, folded, or corrugated porous material layer that is wound into a cylindrical shape by rolling up of the laminar or layered precursor, and then carbonized or pyrolyzed while in the rolled-up form. The exemplary cylindrical supporting body resulting therefrom may comprise a porous material layer rolled up and having a spiral or snail-like in cross section, whereby spaces or channels may extend between the wound layers, approximately parallel to the axis of the cylinder. In such wound bodies, the cross section perpendicular to the cylinder axis may provide a surface that provides a low flow resistance. Similarly, two or more material layer precursors may be stacked, rolled up, and subsequently carbonized or pyrolyzed to form a supporting body. FIGS. 2A and 2B illustrate exemplary cylindrical rolled supporting bodies. The wound bodies may also be produced from one or more alternating layers of corrugated and smooth sheet materials, wherein the intervening smooth sheet prevents the corrugated ridges and troughs from sliding into each other when the multilayer precursor is rolled up.

In further exemplary embodiments of the present invention, the supporting bodies may optionally be modified in order to provide desirable physical and/or chemico-biological properties for certain uses. The supporting bodies may be at least partially hydrophilically, hydrophobically, oleophilically, or oleophobically modified on their interior and/or outer surfaces, for example by fluoridization, parylenization, by coating or impregnation of the supporting bodies with adherence-promoting substances, nutrient media, polymers, and the like.

The porous supporting body may comprise a modular structure that is created, for example, by carbonization of a correspondingly embossed and folded sheet material on the basis of paper, textile, or polymer film, such as described in International Patent Publication WO 02/32558.

In one exemplary embodiment of the present invention, the outer surface of the porous carbon-based body may be at least partially in direct contact with a semipermeable separating layer that may be essentially impermeable to the catalytic units and the reaction products, and which may be at least partially permeable to the reaction medium and the reaction educts, and optionally the remaining outer surface of the supporting body not in contact with the semipermeable separating layer may be sealed. This exemplary embodiment of the present invention has the advantage that the catalytic units and the reaction products may be inhibited or prevented from leaving the catalyst unit by the semipermeable separating layer and the sealing, however, mass transfer of the educts and the reaction medium may be permitted via the semipermeable separation layer. Thus, the catalytic units may be provided with reaction educts, but the products can be retained and may be separated from the catalyst unit in a later operating step. Furthermore, the catalytic units may be protected from discharging from the supporting body in response to such effects as, for example, application of mechanical loads, thereby avoiding potential harmful environmental impact.

This exemplary embodiment of the present invention may further allow for the immersion of several catalyst units in a reaction mixture comprising the reaction medium and the reaction educts, wherein each catalyst unit may comprise different catalytic units, without a mixing of the different products occurring. It may also be employed with different enzymes that may be active in the same nutrient solution. The corresponding catalyst units that can be loaded with different enzymes may, for example, be immersed in a single nutrient medium for active agent production and later be taken from the nutrient medium and opened for removal of active agents. The catalyst units may optionally be designed in such a way that they have to be destroyed for active agent removal, or such that they may be reversibly opened and closed. If the catalyst units can be reversibly opened and closed, they may be cleaned, sterilized, and reused after active agents are removed, for example, by means of extraction.

In an alternative exemplary embodiment of the present invention, the outer surface of the carbon-based porous body may be at least partially in direct contact with a semipermeable separating layer that is essentially impermeable to the catalytic units and may be at least partially permeable to the reaction medium as well as to the reaction educts and products, and, optionally, the remaining outer surface of the supporting body not in contact with the semipermeable separating layer may be sealed. This exemplary embodiment has the advantage that the catalytic units may be inhibited or prevented from leaving the supporting material by the semipermeable separating layer and the sealing, whereas some mass transfer via the semipermeable separating layer may occur. Thus, the catalytic units may be provided with reaction educts, and reaction products may be withdrawn continuously. As described above, the catalytic units may be protected from discharging from the supporting body, which could otherwise lead to potential harmful environmental effects.

Reaction educts and products may diffuse in response to a concentration gradient that can build up between the interior of the catalyst unit (within the optional semipermeable separating layer) and the exterior space (which lies outside of the optionally present semipermeable separating layer). Such species may diffuse through the optional semipermeable separating layer, either into the interior of the catalyst unit or out of the catalyst unit and into the exterior space. The diffusion path may comprise a laminar boundary film on the outer surface of the catalyst unit or the optionally present semipermeable separating layer. Within the porous body, a further mass transport may also occur via diffusion.

A concentration gradient between the interior and exterior spaces of the catalyst unit may be maintained by continuous educt feed and, optionally, by product withdrawal via convection in the exterior space. Mass transport rates may increase in the presence of turbulent flow having increasing Re number, whereas the laminar boundary film on the outer surface of the catalyst unit may tend to be thinner.

The semipermeable separating layer may be a polymer membrane comprising epoxy resins, phenolic resin, polytetrafluoroethylene, polyacrylonitrile copolymer, cellulose, cellulose acetate, cellulose butyrate, cellulose nitrate, viscose, polyetherimide, poly(octyl methyl silane), polyvinylidene chloride, polyamide, polyurea, polyfuran, polycarbonate, polyethylene, polypropylene, and/or copolymers thereof, and the like.

The semipermeable separating layer may comprise carbon fiber, activated carbon, pyrolytic carbon, single-wall or multi-wall carbon nanotubes, carbon molecular sieves, or carbon-containing material deposited by means of CVD or PVD.

Alternatively, the semipermeable separating layer may be a ceramic membrane comprising glass, silicon dioxide, silicates, aluminum oxide, aluminum silicates, zeolites, titanium oxides, zirconium oxides, boron nitride, boron silicates, SiC, titanium nitride, combinations thereof, and the like.

The outer surface of the porous carbon-based supporting body that is not in contact with the semipermeable separating layer may be sealed. The sealing may be accomplished through an impermeable separating layer. This impermeable separating layer may be comprised of the same materials as the semipermeable separating layer and differ from the semipermeable separating layer merely by the pore size. Alternatively, other materials may be used for sealing the supporting body such that essentially no mass transfer takes place between the interior of the body and the exterior space, except via the semipermeable membrane. The sealing may be reversible or irreversible. Irreversible in this context may be understood to mean, for example, that the catalyst unit may have to be destroyed to removal reaction products from within the porous supporting body.

The porous supporting bodies may have a diameter of up to 1 m, preferably up to about 50 cm, or more preferably up to about 10 cm. For some applications, it may be advantageous to provide exemplary catalyst units having smaller diameters to keep the diffusion paths in the interior space of the porous body short. For other applications it may be advantageous to choose catalyst units having larger diameters.

The porous carbon-based bodies may be produced using conventional sintering techniques and methods. In certain exemplary embodiments of the present invention, the porous body may be produced from pyrolyzable organic materials. Subsequently, and preferably prior to or after the introduction of the catalytically active units, the supporting bodies may optionally be provided with a suitable semipermeable separating layer on the outer surface, and they may further be optionally sealed. Semipermeable separating layers may comprise carbon fiber, activated carbon, pyrolytic carbon, single-wall or multi-wall carbon nanotubes, carbon molecular sieve, or carbon-containing material deposited via CVD or PVD procedures.

In another exemplary embodiment of the present invention, porous bodies comprising a semipermeable separating layer may be produced in one step. The production of such porous bodies is described, for example, in German Patent Application DE 103 35 131, and in International Patent Application PCT/EP04/00077.

In certain exemplary embodiments of the present invention, the catalyst unit may be produced by the following:
a) providing a porous carbon-based supporting body as described above, the outer surfaces of which may optionally be in direct contact with a semipermeable separating layer;
b) contacting the porous supporting body with a solution, emulsion, or suspension comprising catalytic units to effect an incorporation of the catalytic units in the porous body;

c) removing the solvent, emulsion, or suspension; and, optionally,
d) applying a further semipermeable separating layer onto, or sealing the remaining outer surface of, the porous supporting body that is not in contact with the semipermeable separating layer.

The supporting body may be immersed in a solution, emulsion, or suspension comprising catalytic units for a period of time of about 1 second to 90 days to allow the catalytic units to diffuse into the porous body and adhere to it.

The porous supporting bodies loaded with the catalytic units produced in such a manner may comprise 10⁻⁵% to 99% by weight of catalytic units, such as metal catalysts, based on the total weight of the loaded porous body.

In an exemplary embodiment of the present invention, the outer surface of the porous carbon-based supporting body may be at least partially in direct contact with a semipermeable separating layer that may be essentially impermeable to the catalytic units and the reaction educts, and which may be at least partially permeable to the reaction medium as well as the reaction products, and optionally the remaining outer surface of the supporting body not in contact with the semipermeable membrane may be sealed. The sealing may be reversible, whereby catalyst units may be opened for product removal after reaction has occurred to some degree. After the removal of products, these catalyst units may be cleaned, optionally sterilized, and reused.

Exemplary Reactors

The exemplary catalyst units can be used in reactors for chemical and/or biological reactions, whereas the reactors may be operated continuously or in a batch mode. The exemplary catalyst units may comprise a semipermeable separating layer. Alternatively, catalyst units without a semipermeable separating layer may be installed in a reactor comprising a semipermeable separating layer in a container or housing. In these exemplary embodiments of the present invention, the container or housing may be designed in such a way that the mass transfer between the reaction mixture outside of the container and that within the container can be controlled by the semipermeable separating layer. The semipermeable separating layer may have the same separation properties as a semipermeable separating layer that can be used in direct contact with the outer surface of the porous body as described above.

Batch-operated stirred tank reactors may be used with catalyst units having a semipermeable separating layer or with catalyst units that are located in a container having a semipermeable separating layer that only allows mass transfer therethrough with respect to the educts and the reaction medium. Such stirred tank reactors may be equipped with a stirring device, and optionally with a continuous educt addition device. The exemplary catalyst units may optionally be immersed in the reaction mixture comprising the reaction medium and the educts within a container that optionally comprises a semipermeable separating layer. It may be preferable to immerse comparatively small catalyst units in the reaction mixture if they are inside a container. The container can allow contact between the catalyst units and the reaction mixture, optionally via a semipermeable separating layer, and may further prevent an uncontrolled distribution of the catalyst units within the reactor.

The flow in the reactor volume or regions thereof may be turbulent, whereby the laminar boundary film around the catalyst units may be thin to improve mass flow rates. Strong convection can assist in maintaining concentration gradients, and educts may be added in sufficient amounts to provide appropriate reaction rates and mass balances.

Increasing turbulence (i.e., an increasing Re number) can lead to higher mass transfer rates via the decrease in size of the effective diffusion paths. Shorter diffusion paths and larger concentration gradients tend to lead to higher mass transfer rates between the interior of a catalyst unit and the surrounding exterior space. The overall rate of many reactions can be limited by mass transfer rather than by the intrinsic reaction rate, such that the conversion rate from reactants to products may depend directly upon the mass transport rates. It may be less common that the intrinsic reaction rate is slower than the mass transport, such that the overall reaction rate would be limited by the intrinsic reaction rate and not by mass transfer considerations.

In other exemplary embodiments of the present invention, a continuous reactor process may be used. A continuous process may have the advantage that educts may be continuously fed and products may be continuously withdrawn. In this manner, as described above, a concentration gradient between the interior of a catalyst unit and the surrounding exterior space can be maintained. Catalyst units that do not have a semipermeable separating layer, or those having a semipermeable separating layer that allows for a mass transfer of educts and products, may be preferably used for these exemplary embodiments of the present invention. As an alternative to catalyst units having a semipermeable separating layer, catalyst units that do not have a semipermeable separating layer may be used whereby they may be introduced into the reactor within a container that has a semipermeable separating layer.

Types of reactors that may be used with such continuous reactor processes include, but are not limited to, continuously operated stirred-tank reactors, tubular reactors, or fluid bed reactors.

Continuously operated stirred-tank reactors may comprise an inlet for the educt/reaction medium mixture, an outlet for the product/reaction medium mixture, and a stirring device. The stirring device may be arranged in such a way to provide good flow around the catalyst unit. The fluid flow may preferably be turbulent, thus providing a thin laminar boundary layer. In certain exemplary embodiments of the present invention where a container is not used and the catalyst units can be immersed directly in the reaction mixture, the catalyst units themselves may be designed in such a way that they favorably influence the flow.

The appropriate reactor retention time in such continuous reactor processes may vary according to the reaction being performed, the reaction rate, and other thermophysical properties such as concentration and temperature.

The educt flow may preferably be recycled, and suitable measuring and controlling devices may be provided in order to control process parameters such as, but not limited to, temperature, pH, and nutrient/reactant or educt concentration. Products may be continuously or discontinuously withdrawn from the circulating flow.

In certain exemplary embodiments of the present invention, the catalyst units may be firmly anchored or affixed to one or more locations within the stirred tank, allowed to move freely within the stirred tank in the reaction medium, or be located in a porous container that is immersed in the reaction medium. If the porous bodies of the catalyst units are allowed to move freely in the reaction medium, they may be prevented from leaving the stirred tank at the reactor outlet. To accomplish this, sieves or similar porous sheets or films, for example, may be attached to the outlet. The catalyst units may be provided inside a porous container that is optionally provided with a semipermeable separating layer, whereby the container is immersed in the reaction mixture. This exemplary embodiment of the present invention has the further advantage that the catalyst units may be easily be removed if the stirred tank is needed for other reactions or if a replacement of the catalyst units is necessary.

In a further exemplary embodiment of the present invention, the reactor may be a tubular reactor. Catalyst units that are elongated may be preferably used in this embodiment. Such catalyst units may be arranged freely or bundled in a container within the tubular reactor. At one end of the tubular reactor, the educt/reaction medium mixture may be introduced, and the product/reaction medium mixture is withdrawn at the other end of the tubular reactor. While the reaction mixture flows through the tubular reactor, the diffusion of educts into the porous support bodies of the catalyst units can take place. The reaction may take place primarily within the porous support bodies, and subsequently the products may diffuse out from the porous body back into the reaction medium. The length of the tubular reactor, as well as the flow rate of the reaction medium, and the retention time associated therewith can be chosen using conventional methods that may depend on the reaction being carried out. The tubular reactor may additionally be equipped with flow perturbers to promote a turbulent flow. As described above with respect to continuously operated stirred reactors, fluid flow having higher Re numbers may be desirable in order to reduce the size of the laminar boundary layers, thereby decreasing the length of the associated diffusion paths and increasing the mass transfer rates. Porous supporting bodies of the catalyst units may optionally be shaped to act as flow disturbers. Alternatively, additional formed pieces may be introduced into the tubular flow reactor that serve as flow disturbers.

In a further exemplary embodiment of the present invention, the reactor may be designed as fluid bed reactor. Conventional fluid bed reactors may be used in conjunction with catalyst units comprising porous supporting bodies of appropriate shapes and sizes. The dimensioning and the reactor conditions may be chosen based on the particular reactions being carried out.

In addition to the basic types of reactors described above, modified forms may also be used without departing from the spirit or scope of the present invention.

In other exemplary embodiments of the present invention, the supporting bodies, catalyst units, and reactors may be used in a variety of catalytic applications including, but not limited to: catalyst supports for exhaust emissions from Otto or Diesel engines, particularly three-way catalyst converters and (oxidative) soot filters or particle combustion units; catalytic processes of the chemical production industry, for example in the processes of oxo synthesis, polyolefin polymerization, or oxidation reactions including ethylene to acetaldehyde, p-xylene to terephthalic acid, SO₂ to SO₃, ammonia to NO, ethylene to ethylene oxide, propene to acetone butene to maleic acid anhydride, or o-xylene to phthalic acid anhydride; in dehydrogenation reactions such as the dehydrogenation of ethylbenzene to styrene, isopropanol to acetone, or butane to butadiene; in hydrogenation reactions, such as the hydrogenation of esters to alcohols and aldehydes to alcohols; in fat hardening; in synthesis of methanol or ammonia; in the ammoxidation of methane to hydrocyanic acid or propene to acrylonitrile; or in refining methods for the cracking of distillative residues, for the dehydrosulfurization, in isomerization reactions, for example of paraffins or of m-xylene to o/p-xylene, in the dealkylation of toluene to benzene, in the disproportionation of toluene to benzene/xylenes, as well as in the steam cracking of natural gas or gasoline, and the like.

The supporting catalysts and catalyst units, as well as reactors comprising these supporting bodies, provided in the exemplary embodiments of the present invention, may be well-suited for a variety of high-temperature and high-pressure reactions, including cartridge systems, because of, at least in part, their chemical inertness, mechanical stability, and porosities, as well as the ease of adjusting various component dimensions. In other exemplary embodiments of the present invention, supporting bodies may be provided for use as filler material for distillation columns with low weight, rectification columns, as catalyst supports in air or water purification devices, or in catalytic exhaust gas cleanup.

### EXAMPLE 1

As supporting material for catalytic units, a natural fiber-containing polymer composite with a mass per unit area of 100 g/m² and 110 μm dry layer thickness was rolled up into a formed piece with a length of 150 mm and a diameter of 70 mm. Radially closed flow channels with an average channel diameter of 3 mm were hereby created from the approximately 8 m long flat material by corrugating and, subsequently, this single-layer corrugated structure was rolled up in a transverse direction and fixed. These formed pieces were carbonized under a nitrogen atmosphere at 800° C. over 48 hours, with air being added at the end of the carbonizing step in order to modify the porosity. A weight loss of 61% of the original mass was observed. The resulting material in water has a pH value of 7.4 and a buffer region in the weakly acidic range.

Disks of about 60 mm diameter and 20 mm thickness each of this carbon material had the following properties: a surface to volume ratio of 1,700 m²/m³, a free flow cross section of 0.6 m²/m³ as a result of the open structure, and a flow channel length of 20 mm. There was no pressure loss detected when water was flowed through the structure under the experimental conditions.

### EXAMPLE 2

As supporting material for catalytic units, layers of a natural fiber-containing polymer composite with a mass per unit area of 100 g/m² and 110 μm dry layer thickness were glued together into a formed piece with a length of 300 mm, a width of 150 mm, and a height of 50 mm. Radially closed flow channels with average channel diameters of 3 mm diameter were created from the flat material by corrugating and subsequent lamination of these single-layer corrugated structures, each offset by 90. These formed pieces were carbonized under a nitrogen atmosphere at 800° C. over 48 hours, with air being added at the end of the carbonizing step in order to modify the porosity. A weight loss of 61% of the original mass was observed. The resulting material in water had a pH value of 7.4 and a buffer region in the weakly acidic range.

By means of water jet cutting, cylindrical supporting bodies of this carbon-based material with a diameter of 35 mm and a thickness of 40 mm were produced. These bodies had the following properties: a surface to volume ratio 1,700 m²/m³, a free flow cross section of 0.6 m²/m³ as a result of the open structure, and a flow channel length of 20 mm. There was no pressure loss detected when water was flowed through the structure under the experimental conditions.

### EXAMPLE 3

As supporting material for catalytic units, a natural fiber-containing polymer composite with a mass per unit area of 100 g/m² and 110 μm dry layer thickness was rolled up into a formed piece with a length of 150 mm and a diameter of 70 mm. Radially closed flow channels in S-shaped or wavelike form with an average channel diameter of 3 mm were produced from the flat material by embossing and subsequent corrugating, and, subsequently, this single-layer corrugated structure was rolled up (see Example 1). These formed pieces were carbonized under a nitrogen atmosphere at 800° C. over 48 hours, with air being added at the end of carbonization in order to modify the porosity. A weight loss of 61% of the original mass occurred. The resulting material in water has a pH value of 7.4 and a buffer region in the weakly acidic range.

Disks of about 60 mm diameter and 20 mm thickness each of this carbon material had the following properties: a surface to volume ratio of 2,500 m²/m³, a free flow cross section of 0.3 m²/m³ as a result of the open structure, and a flow channel length of 20 mm. There was no pressure loss detected when water was flowed through the structure under the experimental conditions.

Having thus described in detail several exemplary embodiments of the present invention, it is to be understood that the invention described above is not to be limited to particular details set forth in the above description, as many apparent variations thereof are possible without departing from the spirit or scope of the present invention. The embodiments of the present invention are disclosed herein or are obvious from and encompassed by the detailed description. The detailed description, given by way of example, but not intended to limit the invention solely to the specific embodiments described herein, may best be understood in conjunction with the accompanying Figures.

The foregoing applications, and all documents cited therein or during their prosecution (“appln. cited documents”) and all documents cited or referenced in the appln. cited documents, and all documents or publications cited or referenced herein (“herein cited documents”), and all documents cited or referenced in the herein cited documents, together with any manufacturer's instructions, descriptions, product specifications, and product sheets for any products mentioned herein or in any document incorporated by reference herein, are hereby incorporated herein by reference, and may be employed in the practice of the invention. Citation or identification of any document in this application is not an admission that such document is available as prior art to the present invention.

It is noted that in this disclosure and particularly in the claims, terms such as “comprises,” “comprised,” “comprising” and the like can have the meaning attributed to them in U.S. Patent law; e.g., they can mean “includes,” “included,” “including” and the like; and that terms such as “consisting essentially of” and “consists essentially of” can have the meaning ascribed to them in U.S. Patent law, e.g., they allow for elements not explicitly recited, but exclude elements that are found in the prior art or that affect a basic or novel characteristic of the invention.

What is claimed is:

1. A supporting body comprising:
at least one carbon-based porous material layer that is at least one of rolled up onto itself or arranged to form a cylindrical body such that at least one space capable of supporting flow exists between at least two adjacent sections of the at least one porous material layer; and
at least one immobilized catalytically active unit associated with the at least one material layer.

2. The supporting body of claim 1, wherein the at least one catalytically active unit comprises at least one of an organometallic complex compound, a metal, a metal oxide, an alloy, or an enzyme.

3. The supporting body of claim 1 wherein the at least one material layer comprises a plurality of material layers, and wherein the at least one space is provided between two adjacent of the material layers.

4. The supporting body of claim 2, wherein the at least one space comprises a plurality of channels.

5. The supporting body of claim 4, wherein the plurality of channels extend in a parallel manner.

6. The supporting body of claim 4, wherein the channels have an average channel diameter in the range of about 1 nm to about 1 m.

7. The supporting body of claim 4, wherein the channels have an average channel diameter in the range of about 1 nm to about 10 cm.

8. The supporting body of claim 4, wherein the channels have an average channel diameter in the range of about 10 nm to 10 mm.

9. The supporting body of claim 4, wherein the channels have an average channel diameter in the range of about 50 nm to 1 mm.

10. The supporting body of claim 4, wherein the channels have a shape of at least one of a linear shape, a wave-like shape, a meandering shape, or a zigzag-shape.

11. The supporting body of claim 4, wherein at least one of the material layer or a wall of the plurality of channels provided at or near the at least one material layer has an average pore size in the range of about 1 nm to 10 cm.

12. The supporting body of claim 4, wherein at least one of the material layer or a wall of the plurality of channels provided at or near the at least one material layer has an average pore size in the range of about 10 nm to 10 mm.

13. The supporting body of claim 4, wherein at least one of the material layer or a wall of the plurality of channels provided at or near the at least one material layer has an average pore size in the range of about 50 nm to 1 mm.

14. The supporting body of claim 1, wherein the at least one carbon-based porous material layer is produced by carbonization of a sheet material that is at least one of structured, rolled, embossed, pre-treated, or folded.

15. The supporting body of claim 14, wherein the sheet material comprises at least one of fiber, paper, textile, or polymer material.

16. The supporting body of claim 1, wherein the supporting body is at least one of arranged in a housing, arranged in a particular container, or arranged on a particular container.

17. The supporting body of claim 16, wherein the particular container comprises at least one of a flask, a bottle, a chemical reactor, a biological reactor, a stirred reactor, a fixed bed reactor, a fluid bed reactor, or a tubular reactor.

18. The supporting body of claim 1, further comprising at least one of activated carbon, sintered activated carbon, amorphous carbon, crystalline carbon, semicrystalline carbon, graphite, pyrolytically produced carbon-containing material, carbon fiber, carbides, carbonitrides, oxycarbides, oxycarbonitrides of metals, or oxycarbonitrides of nonmetals.

19. The supporting body of claim 1, wherein an average pore size of the at least one material layer is between about 2 Å and 1 millimeter.

20. The supporting body of claim 1, wherein an average pore size of the at least one material layer is between about 1 nm and 400 μm.

21. The supporting body of claim 1, wherein an average pore size of the at least one material layer is between about 10 nm and 100 μm.

22. The supporting body of claim 1, wherein an outer surface of the supporting body is at least partially in a direct contact with a semipermeable separating layer that is essentially impermeable to at least one catalytically active unit.

23. The supporting body of claim 22, wherein the semipermeable separating layer comprises a polymer membrane that further comprises at least one of an epoxy resin, a phenolic resin, PTFE, a polyacrylonitrile copolymer, cellulose, cellulose acetate, cellulose butyrate, cellulose nitrate, viscose, polyetherimide, poly(octyl methyl silane), polyvinylidene chloride, a polyamide, a polyurea, a polyfuran, a polycarbonate, polyethylene, polypropylene, or a copolymer of any of the preceding.

24. The supporting body of claim 22, wherein the semipermeable separating layer comprises at least one of a plurality of carbon fibers, activated carbon, pyrolytic carbon, single-wall carbon nanotubes, multi-wall carbon nanotubes, a carbon molecular sieve, a carbon-containing material deposited by CVD, or a carbon-containing material deposited by PVD.

25. The supporting body of claim 22, wherein the semipermeable separating layer comprises a ceramic membrane that further comprises at least one of glass, silicon dioxide, silicates, aluminum oxide, aluminum silicate, a zeolite, titanium oxide, zirconium oxide, boron nitride, boron silicate, SiC, or titanium nitride.

26. The supporting body of claim 22, wherein the semipermeable separating layer has a thickness of between about 3 Å and 1 mm.

27. The supporting body of claim 22, wherein the semipermeable separating layer has a thickness of between about 1 nm and 100 μm.

28. The supporting body of claim 22, wherein the semipermeable separating layer has a thickness of between about 10 nm and 10 μm.

29. The supporting body of claim 22, wherein the semipermeable separating layer has an average pore diameter that is between about 3 Å and 1 mm.

30. The supporting body of claim 22, wherein the semipermeable separating layer has an average pore diameter that is between about 1 nm and 100 μm.

31. The supporting body of claim 22, wherein the semipermeable separating layer has an average pore diameter that is between about 10 nm and 10 μm.

32. A reactor for at least one of a chemical or biological reaction comprising at least one supporting body, wherein the at least one supporting body comprises:
at least one carbon-based porous material layer that is at least one of rolled up onto itself or arranged to form a cylindrical body such that at least one space capable of supporting flow exists between at least two adjacent sections of the at least one porous material layer; and
at least one immobilized catalytically active unit associated with the at least one material layer.

33. The reactor of claim 32, wherein an outer surface of the supporting body is at least partially in a direct contact with a semipermeable separating layer that is essentially impermeable to at least one catalytically active unit.

34. The reactor of claim 32, further comprising a chamber located within the reactor, wherein at least a portion of a wall of the chamber comprises a semipermeable separating layer that is essentially impermeable to the at least one catalytically active unit, and wherein the at least one supporting body is configured within the chamber.
